(19) United States
(12) Patent Application Publication
(10) Pub. No.: US 2008/0004631 A1
(43) Pub. Date: Jan. 3, 2008

(54) Circumcision Clamp

(76) Inventor: David R. Tomlinson, Wakefield, RI (US)

Correspondence Address: GREENBERG TRAURIG, LLP, MET LIFE BUILDING, 200 PARK AVENUE, NEW YORK, NY, 10166, US

(21) Appl. No.: 11/768,817

(22) Filed: Jun. 26, 2007

Related U.S. Application Data
(60) Provisional application No. 60/816,798, filed on Jun. 26, 2006.

(51) Int. Cl. A61B 17/32

(52) U.S. Cl. 606/118

(57) ABSTRACT

The present invention is a modification of a conventional circumcision clamp that prevents the use of mismatched parts.

### CROSS REFERENCE TO RELATED APPLICATIONS [0001| The present invention claims benefit of U.S. Provisional Application Ser. No 60/816,798, entitled “SELF-ADJUSTING PRESSURE APPLICATOR” filed on Jun. 26, 2006, which is incorporated herein.

### FIELD OF THE INVENTION

One embodiment of the present invention is related generally to the field of circumcision.

### BACKGROUND OF THE INVENTION

Newborn circumcision is the most commonly performed surgical procedure in the World with an estimated 5 million circumcisions performed annually. Adult circumcisions are also performed routinely around the world. Circumcision has been performed for centuries for both religious and medical reasons. Circumcision instruments should, among other characteristics, be assembled with matching components to avoid possible penile amputation. For example, a conventional Gomco bell/stud can be lifted through the larger mismatched hole of the conventional Gomco plate and the incision can be made across the plate where the blade passes beneath the bell resulting in an amputation or partial amputation of the glans of the penis of the newborn child. In such cases of mismatched parts, the intended and necessary foreskin crushing action is not performed and hemorrhage can result.

### SUMMARY OF THE INVENTION

### According to the invention, there is provided a circumcision clamp, as defined in claims 1-12.

For a better understanding of the present invention, together with other and further objects thereof, reference is made to the accompanying drawings and detailed description.

### BRIEF DESCRIPTION OF THE DRAWINGS

The present invention is illustratively shown and described in reference to the accompanying drawings, in which:

FIG. 1A-D demonstrates the present invention and illustrates the features that pr event the use of mismatched parts.

### DETAILED DESCRIPTION OF THE INVENTION

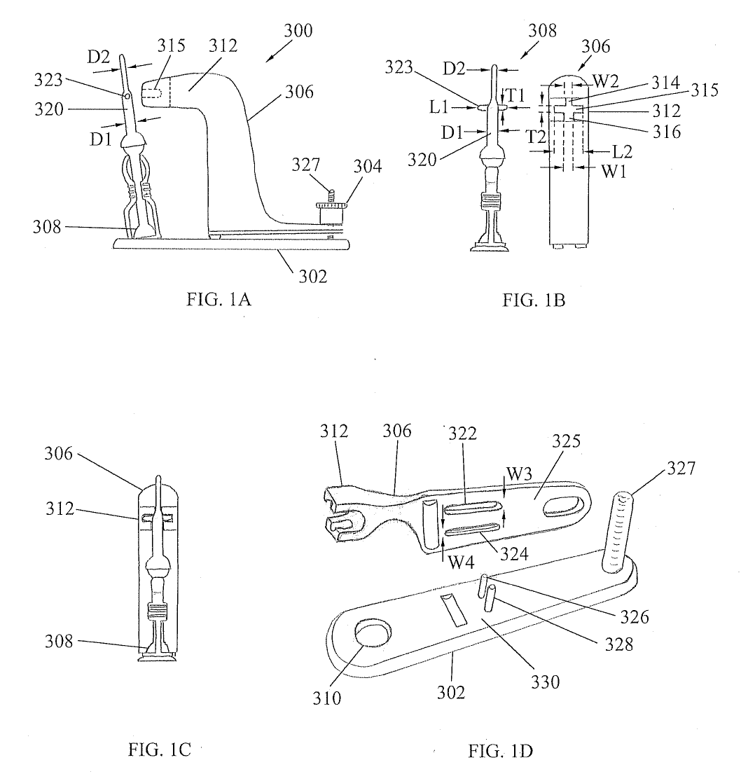

FIG. 1A-D shows one embodiment of the present invention clamp 300. Clamp 300 includes four parts: a plate 302, a nut 304, a yolk 306, and a bell/stud 308. Plate 302 includes a threaded member 327 that call cooperate with nut 304 with complimentary internal threads such that plate 302, yolk 306, and bell/stud 308 are secured in place once assembled as a unit. All four of these parts must be disassembled after each circumcision procedure for cleaning and sterilization and then reassembly to perform the next circumcision. The bell/stud 308 and plate 302 have many different sizes and are accordingly matched together. The present invention ensures that the unit can only be assembled with the appropriately sized parts. Any mismatched parts would prevent assembly and render the device unusable.

Additional embodiments of the matching devices of the present invention to ensure there is a correct fit between the bell/stud 308 and yolk 306 (FIG. 1B-C) includes two slots 314 and 316 (yolk bell/stud matching device) formed in the front portion of the arm 312 of yolk 306. Slots 314 and 316 may have two different widths W1 and W2 that are sized to accept, for example, two diameters D1 and D2 (bell/stud yolk matching device) near the distal end of the shaft 320 of the bell/stud 308. The shaft 320 on bell/stud 308 can only be positioned to work with arm 312 of yolk 306 if, for example, the shaft 320 diameters D1 and D2 were smaller then the widths W1 and W2 of slots 314 and 316, respectively, allowing bell/stud 308 to cooperate appropriately with yolk 306. For example, D1 is complimentary to W1 and D2 is complimentary to D2. It should be appreciated that though D1 is shown as being larger than D2, it is acceptable for D2 to be larger than D1. Also it is within the contemplation of the invention that one or more diameters of various diameter combinations can be formed in the shaft to act as a bell/stud yolk matching device depending, for example, on user needs.

FIG. 2C shows a front view of a matched bell/stud 308 received into the arm 312 of yolk 306, illustrating both the D1/D2 bell/stud yolk matching device and the cross member yolk matching device. However, the invention is not to be limited to the illustrated configuration that is being presented as an example and for convenience. It should be appreciated that that the unique bell/stud yolk matching devices and the yolk bell/stud matching devices can be used singular or in combination (as shown).

Other embodiments of the component matching devices of the present invention to ensure there is a correct fit between bell/stud 308 and yolk 306, shaft 320 can include a cross member 323 (bell/stud yolk matching device) near the distal end of the shaft that includes a complimentary width (or thickness or diameter) and length such that cross member 323 is received into horizontal slot 315 (yolk bell/stud matching device), as shown in FIGS. 1B and C. For example, cross member 323 width T1 and length L1 are smaller than slot 315 width T2 and length L2. It should be appreciated that any dimensions of width and length are acceptable for cross member 323 and slot 315 as long as cross member 323 can be received by slot 315.

In yet another embodiment of the component matching device of the present invention to ensure there is a correct fit plate 330 and yolk 306, the yolk 306 can (FIG. 1D) include at least two slots 322 and 324 on the bottom surface 325 of yolk 306 to form a yolk plate matching device. Plate 302 can include two posts 326 and 328 that extend perpendicular and vertically from the top surface 330 of plate 302 to form a plate yolk matching device. Two posts 326 and 328 may have different diameters that are uniquely sized to fit into the corresponding size slots 322 and 324 of the appropriately sized yolk 306. Slots 322 and 324 have widths W3 and W4 that are slightly larger then the corresponding diameters of posts 326 and 328. By varying the diameters and positions of the posts 326 and 328 on plate 302 and varying the width and position of the slots 322 and 324 between different sized parts, same size parts can be uniquely fitted so that only the correctly sized parts can successfully mate allowing the device to work and that any other combination can not mate, prohibiting the use of the device. The vertical design of the posts 326 and 328 allows a mated yolk 306 to move as is necessary to perform its clamping function. When posts 326 and 328 of a plate 302 mate with the corresponding slots 322 and 324 of yolk 306, any lateral movement of the yolk 306 in relation to the plate 302 is prevented. This ensures the arm 312 maintains its correct position centered over the thru hole 310 of plate 302, providing a further benefit to the user. It should be appreciated that any diameter of post 326, 328 and any widths of slots 322, 324 are acceptable for the plate yolk matching device and the yolk plate matching device, respectively.

Further embodiments of bell/stud 308 are disclosed in U.S. patent application Ser. No. 11/768,808 filed on Jun. 26, 2007, “Self-Adjusting Pressure Applicator”, by David R. Tomlinson, and U.S. patent application Ser. No. 11/571,120 filed on Dec. 21, 2006, “Atraumatic Circumcision Device and Method to Use”, by David R. Tomlinson, which is a U.S. national phase entry of Patent Cooperation Treaty international application serial number PCT/US2005/022404 filed on Jun. 23, 2005, “Atraumatic Circumcision Device and Method to Use”, by David R. Tomlinson, which claims priority to U.S. provisional application Ser. No. 60/583,259 filed on Jun. 25, 2004, “Atraumatic Circumcision Ring and Method”, by David R. Tomlinson, whereby all above listed applications are herein incorporated by reference.

While the disclosure has been described in detail and with reference to specific embodiments thereof, it will be apparent to one skilled ill the art that various changes and modifications can be made therein without departing from the spirit and scope of the embodiments. Thus, it is intended that the present disclosure cover the modifications and variations of this disclosure provided they come within the scope of the appended claims and their equivalents.

What is claimed is:

1. A circumcision clamp comprising:
a plate having a threaded member and a yolk matching device;
a yolk having an arm and a base, wherein the arm includes a bell/stud matching device and the base includes a plate matching device capable of cooperation with the plate yolk matching device;
a bell/stud having a shaft with a yolk matching device capable of cooperation with the yolk bell/stud matching device; and
a nut with internal threads capable of cooperation with the threaded member such that the plate, the yolk, and the bell/stud are secured in place once assembled as a unit,
whereby, the assembled unit will only include matched components.

2. The clamp according to claim 1 wherein the plate yolk matching device comprises two posts extending substantially perpendicular and substantially vertical from a top surface of the plate, wherein the two posts have different diameters.

3 The clamp according to claim 1 wherein the yolk bell/stud matching device comprises two slots.

4. The clamp according to claim 1 wherein the yolk plate matching device comprises two slots.

5. The clamp according to claim 1 wherein the bell/stud yolk matching device comprises the shaft having two diameter s near a distal end of the shaft.

6. The clamp according to claim 1 wherein the bell/stud yolk matching device comprises a cross member.

7. The clamp according to claim 2 wherein the yolk plate matching device comprises two slots having widths complimentary to the two posts.

8. The clamp according to claim 3 wherein the bell/stub yolk matching device comprises the shaft having two diameters near a distal end of the shaft, each diameter being smaller than one complimentary slot of the two slots.

9. The clamp according to claim 3 wherein the bell/stud yolk matching device comprises a cross member having a thickness and length smaller than at least one of the two slots.

10. The clamp according to claim 3 wherein the bell/stud yolk matching device comprises two diameters and a cross member formed in a distal end of the shaft, wherein the two diameters have complimentary widths and the cross member has a complimentary width and a complimentary length such that the two diameters and the cross member are received into the two slots.

11. The clamp according to claim 10 wherein the two diameters have diameters smaller than widths of the two slots.

12. The clamp according to claim 10 wherein the cross member has a width and a length smaller than a width and a length, respectively, of at least one of the two slots.
